(19) United States
(12) Patent Application Publication
(10) Pub. No.: US 2019/0133542 A1
(43) Pub. Date: May 9, 2019

(54) SYSTEMS AND METHODS FOR DATA-DRIVEN RESPIRATORY GATING IN POSITRON EMISSION TOMOGRAPHY

(71) Applicant: The General Hospital Corporation, Boston, MA (US)

(72) Inventors: Quanzheng Li, Cambridge, MA (US); Georges El Fakhri, Boston, MA (US); Mengdie Wang, Boston, MA (US)

(21) Appl. No.: 16/094,144

(22) PCT Filed: Apr. 19, 2017
(86) PCT No.: PCT/US17/28276

§ 371 Date: Oct. 16, 2018

Related U.S. Application Data
(60) Provisional application No. 62/324,659, filed on Apr. 19, 2016.

(51) Int. Cl. A61B 6/00; A61B 6/03

(52) U.S. Cl. CPC A61B 6/5264; A61B 6/469; A61B 6/037; A61B 6/5205; A61N 2005/1052; A61B 6/5247; A61B 6/5288; A61B 6/541; A61B 6/5217

(57) ABSTRACT

Systems and methods for data-driven respiratory gating in positron emission tomography (PET) are provided. In some aspects, a provided method for generating motion information from PET imaging includes receiving time-of-flight (TOF) data acquired using a PET system, and selecting, using at least one image reconstructed from the TOF data, a region of interest (ROI) having tissues subject to motion. The method also includes generating a TOF sinogram mask by projecting an image mask corresponding to the ROI into a sinogram space, and applying the TOF sinogram mask to a TOF sinogram, produced using the TOF data, to identify data in the TOF sinogram associated with motion. The method further includes generating motion information using the data identified.

### CROSS-REFERENCE TO RELATED APPLICATIONS

This application is based on, claims priority to, and incorporates herein by reference in its entirety, U.S. Application Ser. No. 62/324,659, filed Apr. 19, 2016, and entitled “Methods and System For Data-Driven Respiratory Gating.”

### STATEMENT REGARDING FEDERALLY SPONSORED RESEARCH

This invention was made with government support under HLR01EB013293 awarded by the National Institutes of Health. The government has certain rights in the invention.

### BACKGROUND

The field of the disclosure relates to systems and methods for medical and molecular imaging. More particularly, the disclosure relates to systems and methods for respiratory gating in positron emission tomography (“PET”).

In nuclear medicine, radioactive materials incorporated into various substances, such as glucose or carbon dioxide, are administered to patients to gather information about the various biochemical or physiological processes in the body. Positrons emitted through radioactive decay annihilate in tissue, each annihilation creating a pair of photons that propagate in opposite directions. Using PET, tracer activity, including volume distribution and concentration in the body, can then be measured by detecting the emitted photons. Common clinical applications include oncology and cardiology for diagnosing and staging disease, well as monitoring treatment.

Traditional PET systems include one or more rings of radiation detectors that encircle the patient. Coincidence detection circuits connected to paired detectors then record only those photons that are detected within a coincidence timing window. The number of simultaneous events, indicating the number of positron annihilations that occurred along a virtual line joining the two opposing detectors, or line of response (“LOR”), are then counted. An image indicative is then reconstructed by using all annihilation events at each location within a field-of-view. In addition to measuring photon counts, newer generation scanners are also equipped with time-of-flight (“TOF”) capability, which allows for measurement of the difference in arrival time of the photons. This information is used to more accurately determine the location of each annihilation along a LOR. This is because in conventional non-TOF PET reconstruction, the intensity of all voxels associated with an LOR is incremented regardless of position along the LOR. On the other hand, with TOF PET, each voxel intensity is incremented by a probability that the source originated at that voxel.

A major source of artifacts in PET images is motion due to respiratory and cardiac activity. Specifically, physiological activity causes organs, such as heart muscle, lung, or abdominal organs, to change location, shape, or local tissue density, resulting in a complex non-rigid movement patterns, particularly in the thoracic-abdominal region. Also, different organs may move with different amplitudes, and hence their effect on the respiratory signal may differ. For example, respiratory motion may displace the lower lobes of the liver along the cranial-caudal direction between 10 and 14 mm while the diaphragm may be displaced between 20 and 38 mm.

Since the acquisition of PET is typically much longer than the respiratory period, motion limits the spatial resolution that can be achieved in PET imaging. In fact, physical factors, such as detector size, photon non-collinearity and positron range of travel, generally contribute less to a deterioration of spatial resolution, on the order of 1-3 mm, as compared to 5-15 mm due to organ motion. As such, motion artifacts result in significantly lowered resolution, leading to poor detectability of tumors, inaccurate standard uptake value (SUV) calculations, incorrect PET-measured tumor volumes, and reduced accuracy in the localization of PET abnormalities.

Methods for reducing respiration-induced artifacts in PET imaging have included prospective and retrospective gating. In prospective gating, respiratory patterns are used to trigger data acquisition at specific times in the respiratory cycle. On the other hand, in retrospective gating, data acquired over the entire respiratory cycle is separated according to respiration phases, under the assumption that no appreciable movement takes place within each phase. Events accumulated within each phase are then used to reconstruct separate images.

Respiratory patterns for gating can be obtained indirectly from various external monitors, such as thoracic belts, bellows or video monitoring, which measure chest or abdominal wall excursion. Such, external monitoring techniques rely on the assumption that the measured parameter provides an accurate estimate of the respiratory state of the structure being imaged. However, in practice, tissue movement inside the body is complex, and need not coincide with chest or abdominal wall movements.

In other techniques, the acquired PET data is used to estimate the respiratory patterns, without need for additional equipment. In such data-driven approaches, motion profiles are obtained directly by manually selecting and analyzing lines or regions of interest (ROIs) on reconstruction dynamic frames spanning moving boundaries. Alternatively, rather than selecting an ROI in an image to obtain respiratory signals, some have proposed utilizing a corresponding LOR in sinogram space. Yet others have used spectral analysis of PET sinograms to identify data subject to respiratory motion. However, image-based methods are generally more time-consuming because they typically require reconstruction of all images in advance, and need a fine temporal scale to guarantee enough samples for resolving the different respiration phases. Also, such approaches fail for images having low signal-to-noise ratio (SNR).

Therefore, in light of the above, improvements in the performance of respiratory gating for PET imaging are urgently needed.

### SUMMARY

The present disclosure provides methods and systems for improved positron emission tomography (PET) that overcome the drawbacks of previous technologies. In particular, a novel data-driven approach that uses time-of-flight (TOF) PET data to obtain motion information is introduced herein. As will be described, such motion information may then be used for gated acquisition.

In accordance with one aspect of the disclosure, a method for generating motion information from positron emission tomography (PET) imaging is provided. The method includes receiving time-of-flight (TOF) data acquired using a PET system, and selecting, using at least one image reconstructed from the TOF data, a region of interest (ROI) having tissues subject to motion. The method also includes generating a TOF sinogram mask by projecting an image mask corresponding to the ROI into a sinogram space, and applying the TOF sinogram mask to a TOF sinogram, produced using the TOF data, to identify data in the TOF sinogram associated with motion. The method further includes generating motion information using the data identified.

In accordance with another aspect of the disclosure, a system for generating motion information from positron emission tomography (PET) imaging is provided. The system includes a non-transitory computer readable medium having stored therein instructions for generating motion information, and a processor configured to execute the instructions to receive time-of-flight (TOF) data acquired from a subject using a PET system, and reconstruct at least one image using the TOF data. The processor is also configured to generate an image mask based on a selection of a region of interest (ROI) having tissues subject to motion, and generate a TOF sinogram mask by projecting the image mask into a sinogram space. The processor is further configured to apply the TOF sinogram mask to a TOF sinogram, produced using the TOF data, to identify data in the TOF sinogram associated with motion in the TOF sinogram, and generate motion information using the identified data.

In accordance with yet another aspect of the disclosure, a non-transitory, computer-readable storage medium having stored thereon instructions is provided. The instructions, when executed by a processor, cause the processor to generate a report indicative of motion information, and include accessing time-of-flight positron emission tomography (TOF-PET) data acquired from a subject using a positron emission tomography (PET) system, reconstructing one or more images from the TOF-PET data, and selecting, using at least one of the images, a region of interest (ROI) in which tissues are subject to motion. The instructions also include generating a TOF sinogram mask by projecting an image mask corresponding to the ROI into a sinogram space, producing a TOF sinogram using the TOF-PET data, and applying the TOF sinogram mask to TOF sinogram to identify data associated with motion in the TOF sinogram. The instructions further include generating motion information using the identified data, and generating a report indicative of the motion information.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a flowchart setting forth the steps of a process, in accordance with aspects of the present disclosure.

FIG. 2 is a diagram of an example system, for use in accordance with aspects of the present disclosure.

FIG. 3 is a schematic diagram depicting a positron emission tomography system (PET), in accordance with certain embodiments of the present disclosure.

FIG. 4 is a flow diagram showing steps of a process, in accordance with the present disclosure.

FIG. 5A is an image based on time-of-flight (TOF) PET data, showing signal contribution for a region of interest.

FIG. 5B is another image based on non-TOF PET data, showing signal contribution for a region of interest.

FIG. 6 is a graph illustrating raw and filtered respiratory traces generated using TOF-PET and non-TOF PET data.

FIG. 7 is a graph comparing a respiratory trace obtained in accordance with aspects of the present disclosure and a trace obtained using a bellows monitor.

FIG. 8 is another graph showing respiratory traces obtained with bellows non-TOF-PET data without mask, non-TOF data with mask and TOF data with mask, in accordance with aspects of the present disclosure.

FIG. 9 is yet another graph showing respiratory traces obtained with bellows, non-TOF data with mask and TOF data with mask, in accordance with aspects of the present disclosure.

FIG. 10 is yet another graph showing respiratory traces obtained with bellows, non-TOF data with mask and TOF data with mask.

FIG. 11 shows sagittal views of non-gated and gated images obtained by TOF-PET data-driven gating, in accordance with aspects of the present disclosure.

FIG. 12 is a graph showing the 1D activity profiles from the summed and gated reconstructed images of FIG. 11.

FIG. 13 shows reconstructed sagittal images comparing TOF and non-TOF data approaches.

FIG. 14A is a graph showing center of mass displacement for the present approach in comparison to other approaches.

FIG. 14B is another graph showing center of mass displacement for the present approach in comparison to other approaches.

### DETAILED DESCRIPTION OF THE INVENTION

Correcting positron emission tomography (“PET”) images for respiratory motion is important because motion-induced artifacts can significantly reduce image resolution, hindering the visibility of structures that could otherwise be seen using current PET technologies. In some cases, poor image resolution may result in a different diagnostic outcome. Hence, rather than obtaining respiratory patterns, and other motion information, indirectly using various external monitors, the present disclosure introduces a novel data-driven approach. As will be described, in some aspects, systems and methods disclosed may utilize time-of-flight (TOF) PET data to generate motion information for respiratory gating in PET.

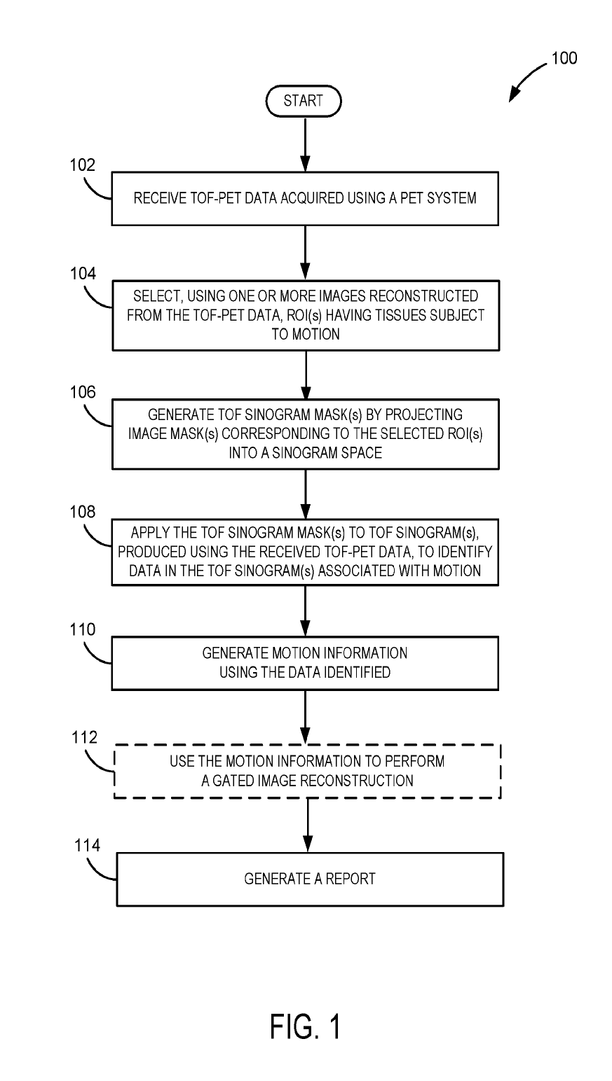

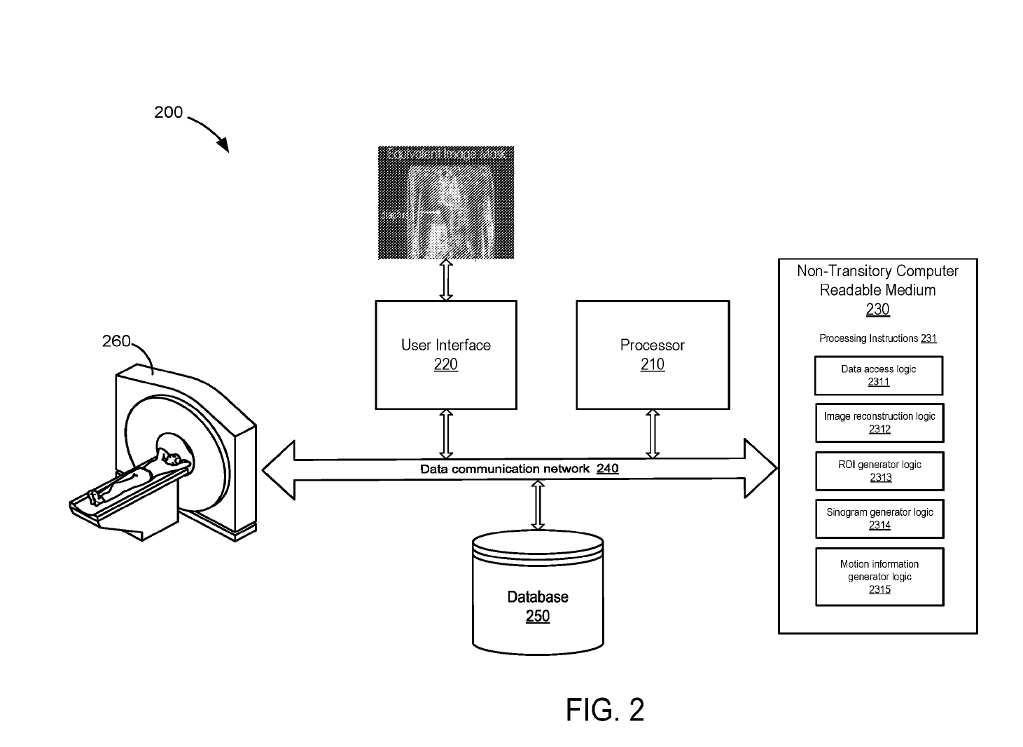

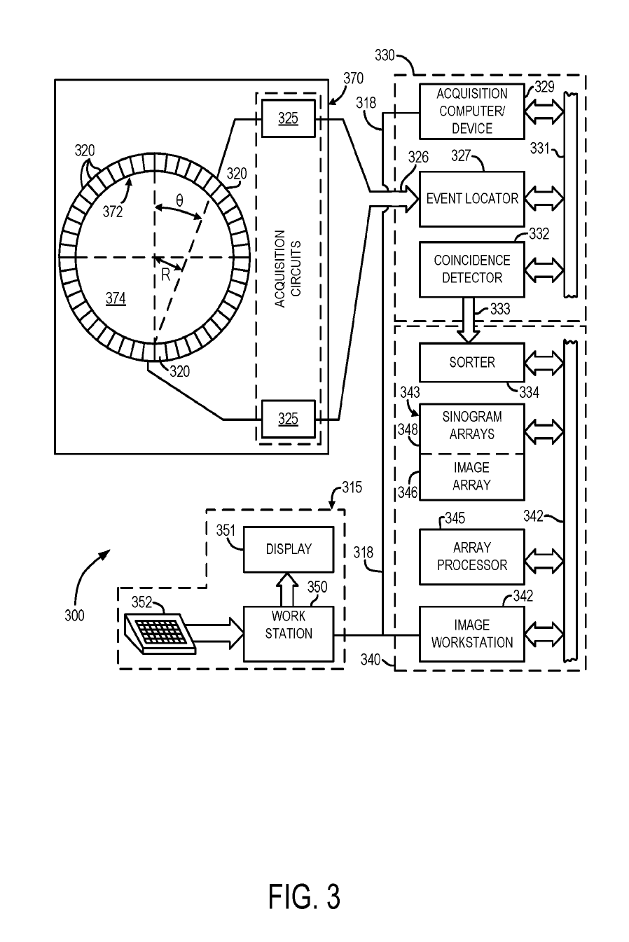

Referring specifically to FIG. 1, a flowchart setting forth the steps of a process 100, in accordance with aspects of the present disclosure, is shown. The process 100 may be carried out using any suitable devices or systems, such as the systems described with reference to FIGS. 2 and 3. In some aspects, the process 100 may be implemented as a program or instructions stored in non-transitory computer readable media executable by a processor or computer.

The process 100 may begin at process block 102 with receiving TOF-PET data, and other data. Examples of other data may include computed tomography (CT) data, magnetic resonance (MR) data, and so forth. In some aspects, the data may be accessed at process block 102 from a memory, a database, or other storage location. Alternatively, or additionally, TOF-PET data may also be acquired by performing a PET scan using a PET system. By way of example, the TOF-PET data received at process block 102 may be list-mode data including information about coincidence events and timing tags. In some aspects, TOF-PET data may be assembled, sorted, or otherwise processed at process block 102. For example, the data may be binned according to event timings, detector pairs, and so forth. For instance, the TOF-PET data may be separated into time bins each having a duration of approximately 100 ms, although other values may be possible.

Then at process block 104, one or more regions of interest (ROIs) having tissues subject to motion may be selected or identified using one or more images. The images may be reconstructed from the received or accessed TOF-PET data, as well as other imaging data, using various reconstruction algorithms. In some aspects, the reconstructed images may include two-dimensional (2D) or three-dimensional (3D) PET images, as well as a time series of PET images. ROIs may be selected at process block 104 manually by a user, by providing a selection via a user interface or input. Alternatively, or additionally, ROIs may be automatically identified, using various segmentation techniques known in the art. In some aspects, one or more image masks corresponding to the selected ROI(s) may also be constructed and optionally displayed.

ROIs selected at process block 104 may include various tissues or organs, or portions thereof, including tissues or organs associated with a thoracic or abdominal region, and elsewhere. For example, selected ROIs may include lung tissue, heart tissue, liver tissue, diaphragm tissue, and others. Herein, an ROI may generally refer to one-dimensional (1D), two dimensional (2D) or three-dimensional (3D) regions. In some aspects, selected ROIs may be shaped to be rectangular, square, cuboid or cubic. To this end, at least one dimension, for example a longest dimension, of the selected ROIs may extend in an axial direction, a transaxial direction, or transverse direction. Here, the directions may be determined based on a pre-defined coordinate system of a subject or an imaging system. Selected ROIs may also have other shapes. In some aspects, the ROIs may be selected in a manner that captures the largest movement or displacement of target tissues or organs.

For each selected ROI, a TOF sinogram mask may be generated, as indicated at process block 106. This step includes projecting the image masks corresponding to the selected ROIs into a sinogram space. In some aspects, the image masks may be representative of a specific volume of interest. The TOF sinogram masks may then be applied to TOF sinogram(s), produced using the received or accessed TOF-PET data, as indicated by process block 108. The TOF sinograms may be 2D or 3D sinograms. Applying the masks allows for identifying data in the TOF sinograms that is associated with motion. In this manner, location-sensing sinograms (LSS) may be generated, representing event data indicative of motion. As described, this allows for the selection of data specific to motion, without contamination from other tissues or organs.

Then, at process block 110, motion information may be generated using the data identified at process block 108. As will be described, this may include generating one or more waveforms or traces, such as respiratory waveforms, from the data specific to motion using a center of mass (COM) algorithm. In some aspects, a single slice rebinning (SSRB) algorithm may also be performed at process block 110. In addition, generated waveforms may be filtered using a band-pass filter, such as a Gaussian band-pass filter, as well as other filters to eliminate undesired noise or frequency components therein. In some aspects, in generating motion information, various motion waveforms or traces may be analyzed at process block 110 to determine amplitudes of motion, frequencies of motion, and directions of motion.

As indicated by process block 112, the generated motion information may be used to perform a gated image reconstruction using the received or accessed TOF-PET data. A report, in any form, may then be generated at process block 114 and provided via a user interface or display. In some implementations, the report may include a visual representation of generated motion information, as well as other information. For example, the report may include one or more time traces or waveforms indicative of motion, such as respiratory waveforms. The report may also highlight specific tissues or organs subject to motion, as well as indicate directions or extent of movement. The report may further include one or more reconstructed images, or gated images, as well as images showing motion in substantially real-time. In some aspects, the report may be provided in the form of electronic signals or instructions to a treatment or imaging device or system. For instance, generated motion information in the report may be used by the device or system to adapt a treatment or an imaging protocol.

Turning now to FIG. 2, a diagram of an example system 200, in accordance with aspects of the present disclosure, is shown. In general, the system 200 may include a processor 210, a user interface 220, and a non-transitory computer readable medium 230. In particular, the user interface 220 may include various input elements for receiving user input and selections, such as a mouse, keyboard, touchscreen, and the like. The user interface 220 may also include various output elements, such a display and the like, which may be used to provide a report.

In some implementations, the system 200 can be a computer, workstation, a network server, a mainframe or any other general-purpose or application-specific computing device. The system 200 may also be a portable device, such as a mobile phone, laptop, tablet, personal digital assistant (“PDA”), multimedia device, or any other portable device.

The system 200 may operate as part of, or in collaboration with, one or more computers, devices, machines, mainframes, servers, cloud, the internet, and the like. As such, the system 200 includes a data communication network 240 configured to, not only facilitate communication between the processor 210, the user interface 220, the non-transitory computer readable medium 230, but also enable communication with external devices and systems. For example, as shown in FIG. 2, the data communication network 240 provides communication and data exchange with a database 250 (i.e. a PACS server), an imaging system 260 (i.e. a PET system), or other system or data storage location.

In addition to performing various processing tasks for operating the system 200, the processor 210 may also be configured or programmed to carry out steps for generating motion information, in accordance with aspects of the present disclosure. To this end, the processor 210 may be configured to execute a program or carry out instructions stored, for instance, in the non-transitory computer readable medium 230. In particular, the processor 210 may be configured to receive, or access, and then analyze TOF-PET data or images, as well as other data or images, such as computed tomography (CT), magnetic resonance (MR), ultrasound (US) data or images. The processor 210 may also be configured to control the imaging system 260 to perform an acquisition, or to retrieve data or images therefrom. In addition, the processor 210 may be configured to process the received, accessed, or acquired imaging data, and generate therefrom one or more images, including attenuation-corrected images, using Filtered Back-Projection reconstruction, iterative reconstruction or other reconstruction techniques.

In some aspects, the processor 210 may be configured to analyze reconstructed, received or accessed images, to select or identify various ROIs. In particular, the processor 210 may be configured to select ROIs having tissues, organs, or portions thereof, which are subject to motion. For example, selected ROIs may include lung tissues, heart tissues, liver tissues, diaphragm tissues, and others. As described, selected ROIs may extend in axial, transaxial, or transverse directions. In identifying or selecting the ROIs, the processor 210 may utilize various segmentation algorithms as well as image registration and analysis techniques. Additionally, or alternatively, the processor 210 may also utilize input provided by a user via the user interface 220.

Based the selected ROIs, the processor 210 may generate TOF sinogram masks. To do so, the processor 210 may transform image masks associated with the selected ROIs into TOF sinogram masks by projecting them from an image space into a sinogram space. The TOF sinogram masks may then be applied to TOF sinograms to identify data therein associated with motion. As described, TOF sinogram, which may include 3D TOF sinograms, may be generated by the processor 210 using received, accessed or acquired TOF-PET data. By using the TOF sinogram masks, data specifically associated with motion may then identified and subsequently used to generate motion information. In some aspects, the processor 210 may be configured to apply a SSRB algorithm to the identified data. The processor 210 may then apply a COM algorithm to generate the motion information. As described, motion information may be in the form of traces or waveforms. As such, the processor 210 may also be configured to filter these generated waveforms, for example, by applying a band-pass filter, such as a Gaussian band-pass filter. In some aspects, the processor 210 may be configured to analyze various raw or filtered waveforms or traces to determine amplitudes of motion, frequencies of motion, and directions of motion.

Motion information, along with other information, may be provided to a user in a report generated by the processor 210. The processor 210 may further provide the report to various external systems or devices. In some aspects, the processor 210 may be further configured to use the motion information to perform a gated image reconstruction using the TOF-PET data. For instance, the gated image reconstruction may be performed using an ordered subset expectation maximization (OSEM) reconstruction, such as point spread function (PSF) OSEM reconstruction. Attenuation, and other corrections, may be carried out by the processor 210 in the gated image reconstruction.

The system 200 may operate autonomously or semi-autonomously and perform a variety of functions and processing tasks. In this regard, the system 200 may integrate a variety of software and hardware capabilities and functionalities. As described, the processor 210 of the system 200 may execute processing instructions 231 stored in the non-transitory computer readable medium 230, and generate a report in accordance with aspects of the present disclosure. As shown in FIG. 2, in some implementations, the processing instructions 231 may include:

a data access logic 2311 configured to retrieve or access data, including TOF-PET data acquired using a positron emission tomography (PET) system;

an image reconstruction logic 2312 configured to reconstruct one or more images using TOF-PET data, and optionally use motion information during the reconstruction;

an ROI generator logic 2313 configured to select or identify ROIs subject to motion using reconstructed or provided images;

a sinogram generator logic 2314 configured to generate TOF sinogram masks by projecting selected or identified ROIs into a sinogram space, as well as produce TOF sinograms using TOF-PET data;

a motion generator logic 2315 configured to apply TOF sinogram masks to generated TOF sinograms to identify therein data associated with motion, and generate motion information using the identified data.

In addition to executing instructions in the non-transitory computer readable medium 230, the system 200 may also may alternatively or additionally receive instructions from a user via the user interface 220, or any source logically connected to the system 200, such as another networked computer, device or server.

Turning now to FIG. 3, a schematic diagram depicting a positron emission tomography system (PET) 300, in accordance with certain embodiments of the present disclosure, is shown. Although PET system 300, as represented in the example of FIG. 3, can be implemented as a stand-alone imaging system, in accordance with some aspects of the present disclosure, it may be appreciated that PET system 300 may also utilized in combination with other imaging systems. For example, PET system 300 may be integrated into a multi-modality, or hybrid, imaging system, such as a PET/CT system, or a PET/MR system. In some aspects, raw or processed PET data, or images, generated using PET system 300 may be directly used to generate photon attenuation maps, and/or attenuation-corrected images. In other aspects, raw or processed PET data, or images, may be combined with information from other raw or processed data or images, such as CT or MR data or images, to generate photon attenuation maps, and/or attenuation-corrected images.

As illustrated in FIG. 3, PET system 300 includes a gantry 370, which supports a detector ring assembly 372. The detector ring 372 includes detector units 320. The signals produced by the detector units 320 are then received by a set of acquisition circuits 325, which produce digital signals indicating the line of response and the total energy. These signals are sent through a communications link 326 to an event locator circuit 327. Each acquisition circuit 325 also produces an event detection pulse (“EDP”) which indicates the exact moment the scintillation event took place.

The event locator circuits 327 form part of a data acquisition processor 330, which periodically samples the signals produced by the acquisition circuits 325. The processor 330 has an acquisition CPU 329 which controls communications on local area network 318 and a backplane bus 331. The event locator circuits 327 assemble the information regarding each valid event into a set of digital numbers that indicate precisely when the event took place and the position of a scintillator crystal which detected the event. This event data packet is conveyed to a coincidence detector 332, which is also part of the data acquisition processor 330.

The coincidence detector 332 accepts the event data packets from the event locators 327 and determines if any two of them are in coincidence. Coincidence is determined by a number of factors. First, the time markers in each event data packet must be within a preset time of each other, and second, the locations indicated by the two event data packets must lie on a straight line. Events that cannot be paired are discarded, but coincident event pairs are located and recorded as a coincidence data packet.

The coincidence data packets are conveyed through a link 333 to a sorter 334 where they are used to form a sinogram. The sorter 334 forms part of an image reconstruction processor 340. The sorter 334 counts all events occurring along each projection ray (R, θ) and organizes them into a two dimensional sinogram array 348 which is stored in a memory module 343. In other words, a count at sinogram location (R, θ) is increased each time a coincidence data packet at that projection ray is received.

The image reconstruction processor 340 also includes an image CPU 342 that controls a backplane bus 341 and links it to the local area network 318. An array processor 345 also connects to the backplane 341 and it reconstructs an image from the sinogram array 348. The resulting image array 346 is stored in memory module 343 and is output by the image CPU 342 to the operator work station 315.

The operator work station 315 includes a CPU 350, a display 351 and a keyboard 352. The CPU 350 connects to the network 218 and it scans the keyboard 252 for input information. Through the keyboard 352 and associated control panel switches, the operator can control the calibration of the PET scanner and its configuration. Similarly, the operator can control the display of the resulting image on the display 351 and perform image enhancement functions using programs executed by the work station CPU 350.

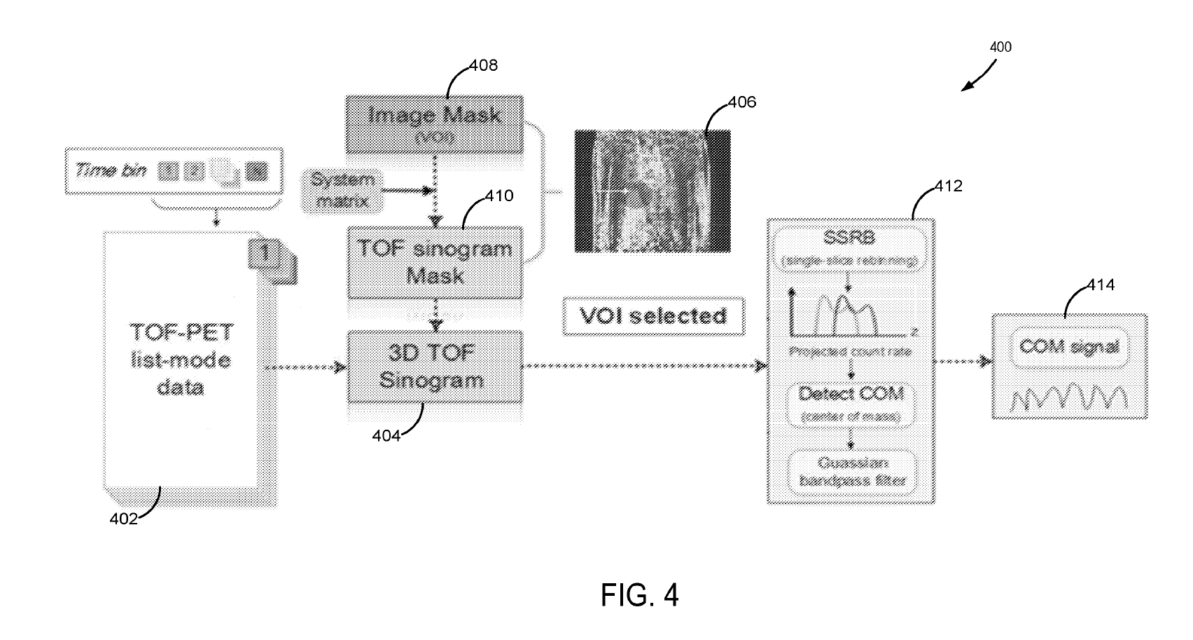

Turning to FIG. 4, a flow diagram, illustrating the steps a process 400 for generating motion signals for respiratory gating according to the present disclosure, is shown. As described, the present data-driven approach may be used to extract respiratory motion information by taking advantage of better localization information contained in TOF-PET data. Hence, as illustrated in FIG. 4, list-mode TOF-PET data 402, consisting of coincidence events and timing tags, is utilized in the process 400. In some aspects, the list-mode TOF-PET data 402 may be separated into time intervals, or frames, approximately 100 ms in duration, although other durations may be possible. Data in the list-mode TOF-PET data 402 stream may be combined into histogrammed projection arrays, or sinograms. Hence, as indicated, 3D TOF sinograms 404 may be generated using the TOF-PET data 402.

In order to provide a direct estimate of the respiratory state using the data, at least one ROI having tissues subject to respiratory motion can be selected. As shown in the example of FIG. 4, selection may be performed using at least one reconstructed image 406 based on the acquired list-mode TOF-PET data 402 as well as other data. Since the diaphragm typically moves consistently with respiration, an area (ROI) or volume (VOI) around the diaphragm may be selected. As described, a user may provide an indication that defines or selects the ROI or VOI. Alternatively, an automated or semi-automated segmentation algorithm may be utilized. It may be appreciated that other tissues or organs may also be selected or defined, and discussion that follows is applicable to any selected ROI or VOI.

The selected ROI or VOI may then be used to generate an image mask 408. By way of example, the image mask 408 may have non-zero values (for example, 1) for pixels or voxels within the selected ROI or VOI, and zero elsewhere. The image mask 408 may then be used to generate a localization-sensing, TOF sinogram mask 410. To do so, the image mask 408 may be projected in sinogram space using the following system matrix equation:

$${p}_{i , tof} = {\sum }_{j = 1}^{N} {a}_{i , j , tof} {V}_{j} , {V}_{j} = \{ \begin{matrix}1 , & j ∈ D \\ 0 , & j ∉ D\end{matrix} \qquad ( 1 )$$

where a_{i,j,tof} may the coefficient of the system matrix for LOR i, voxel j and TOF bin tof, Vⱼ may be the mask vector, and D may be the diaphragm. Applying the TOF sinogram mask 410 to the 3D TOF sinogram 404, obtained from TOF-PET list-mode data as indicated in FIG. 4, then generates a location-sensing sinogram that identifies data in the 3D TOF sinogram 404 associated with motion. An analysis 412 of the identified data may then carried out to determine motion information.

In some implementations, a center of mass (COM) signal 414 may be obtained by applying a center of mass (COM) algorithm to the identified data. For example, the COM signal 414 may represent a respiratory signal or waveform. In some aspects, oblique coincidence events, associated with the 3D location-sensing sinogram may be rebinned using a single Slice Rebinning (SSRB) algorithm to lower dimensional format. For example, coincidence events may be rebinned into approximately 109 or other number of transverse sinograms. As such, the COM algorithm may then be based on a direct estimation of the motion inside the field of view. As such, the coincidence counting rate per frame may be determined by the processed sinogram as a function of the axial coordinate (slice number). As such, SSRB enables an axial assignment of coincidence events along the scanner's z-axis.

Activity inside the thorax generally tracks with respiratory motion. As a result, the axial component of a sinogram contains information about the respiratory phase. To extract this information, the axial center of mass may be computed as a function of time frame using the following:

$${Z}_{com} = \frac{{\sum }_{k = 1}^{{N}_{t}} {P}_{i ( k ) , tof ( k )} {z}_{k}}{{\sum }_{k = 1}^{{N}_{t}} {P}_{i ( k ) , tof ( k )}} \qquad ( 2 )$$

where i(k) and tof(k) may be the line of response and time bin which measured event k may belong to, respectively. zₖ may be the slice number of event k, so that the weighted z_{com} for a total of Nₜ events within time frame t may be calculated. In some aspects, if P_{1,tof} goes through both pixel j∈D and pixel k∉D, then the effect of k may be mitigated or avoided by selectively weighting P_{t,tof} along the time bins direction. Masks obtained using Eqn. 1 would then yield much higher SNR for the generated respiratory signal and better performance of respiratory gating.

The COM signal 414 may be generally sinusoidal, due to the cyclical nature of respiratory activity. However, due to statistical fluctuations as well as the translation of the heart during the cardiac contraction, the COM signal may contain various frequency components that are not only at the respiratory frequency, including a strong component due to the heartbeat. Therefore, in some aspects, a Gaussian band-pass filter may be applied to the COM signal 414 to obtain a filtered respiratory signal that provides more accurate respiratory motion information.

In some aspects, respiratory gated reconstruction may be conducted using the respiratory motion information. In order to perform respiratory-gated reconstruction, peak times of respiratory cycles may be determined using the respiratory signals extracted. For instance, after applying a band-pass filter, the peaks of each respiratory cycle may be detected by finding local maxima in the filtered respiratory signals using an adaptive algorithm. As am example, a 5-min frame may be used to perform respiratory phase-gated reconstruction with PSF-OSEM, using either mask-generated or Anzai-generated gating information. Respiratory cycles may be included in the reconstruction if their periods may be within one standard deviation of the mean period duration of this 5-min frame data. Each respiratory cycle may be divided into 6 gates.

As described, the present disclosure recognizes that TOF-PET data may be advantageously utilized to generate location-sensing sinograms to provide more accurate motion information. Hence, TOF-PET data was utilized in the above-described process 400. Since the diaphragm may share the same LOR with the surrounding tissue or organs, which would contribute differently to the signal calculation, these regions would consequently contaminate the extracted signal based on non-TOF data. By contrast, the TOF sinogram mask described herein is able to sense the location along the LOR, and can eliminate the ‘noise’ from other organs by setting zeros at the corresponding time bin within the field of view (FOV).

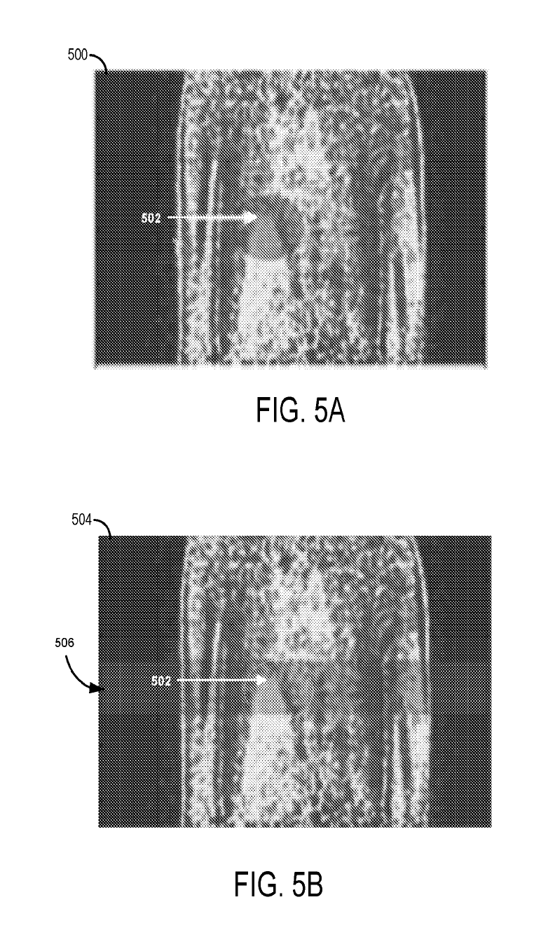

This distinction is illustrated in FIGS. 5A and 5B, whereby an ROI 502 of a partial diaphragm selected on an TOF image 500 includes data that is substantially limited to the ROI 502, as shown in FIG. 5A. By contrast, the same ROI 502 selected on a non-TOF image 504 would include data from a band 506 extending beyond the ROI 502, including signals from other organs and tissues therein.

In addition to descriptions above, specific examples are provided below, in accordance with the present disclosure. These examples are offered for illustrative purposes only, and are not intended to limit the scope of the present invention in any way. Indeed, various modifications in addition to those shown and described herein will become apparent to those skilled in the art from the foregoing description and the following example and fall within the scope of the appended claims.

### EXAMPLE

Data-driven respiratory gating method may be capable of detecting breathing cycles directly from positron emission tomography (PET) data, but may fail at low signal-to-noise ratio (SNR), particularly at low dose PET/CT study. It is recognized herein that time-of-flight (TOF) PET can provide better localization of region of interest (ROI) in sinogram space with improved signal-to-noise ratio (SNR). In order for TOF information to reduce the statistical noise and boost the performance of respiratory gating, a robust data-driven respiratory gating method is herein presented based TOF information. As will be described, respiratory signals can be retrospectively obtained from the ROI-specified TOF-PET data.

Specifically, PET data was acquired in list-mode format and analyzed in sinogram space. The present approach was demonstrated using patient datasets acquired on a PET/CT system. Data-driven gating by center of mass (COM) was successfully performed on PET data with and without TOF information. To assess the accuracy of the data-driven respiratory signal, a hardware-based signal was acquired for comparison. The resulting respiratory-gated images were compared to those obtained using a non-TOF method, highlighting substantial improvements in image quality of the present approach. Specifically, this study showed that retrospectively respiratory gating using TOF sinograms, in accordance with the present disclosure, can provide improved SNR with better resolution, outperforming non-TOF gating techniques.

### Methods

Five minute chest PET/CT scans were acquired from three human subjects approximately 90 min after an injection of 6 mCi 18F-FDG. A Siemens Biograph mCT system (Siemens Medical Solutions USA, Inc.) was used for this study, which included four rings of 48 blocks, each having 13×13 crystals (4.01 mm×4.01 mm×20 mm). List-mode TOF-PET data was acquired, with events being measured with 78 psec time bins. A rebinning into 13 time bins with 312 psec bin width and 580 psec FWHM was performed. For comparison, respiratory patterns were acquired concurrently with the PET data using an Anzai AZ-733 system. Non-TOF PET sinogram data was generated by summing the TOF sinogram data in the TOF bin direction. The emission coincidence rate was 141 kcps, with 36 million prompt events collected within 5 minutes.

Sinogram data was analyzed using a temporal period of 100 ms, and respiratory signals were extracted using an approach as described herein. After band-pass filtering, peaks associated with each respiratory cycle were detected by finding local maxima using an adaptive algorithm. Similar detection was applied to centroids-of-distribution (COD) traces that were generated. An approximately 5-min frame was used to perform respiratory phase-gated reconstruction with PSF-OSEM, using either mask-generated or Anzai-generated gating information. Respiratory cycles were included in the reconstruction if their periods were within one standard deviation of the mean period duration of this 5-min frame data. Each respiratory cycle was divided into 6 gates.

### Results

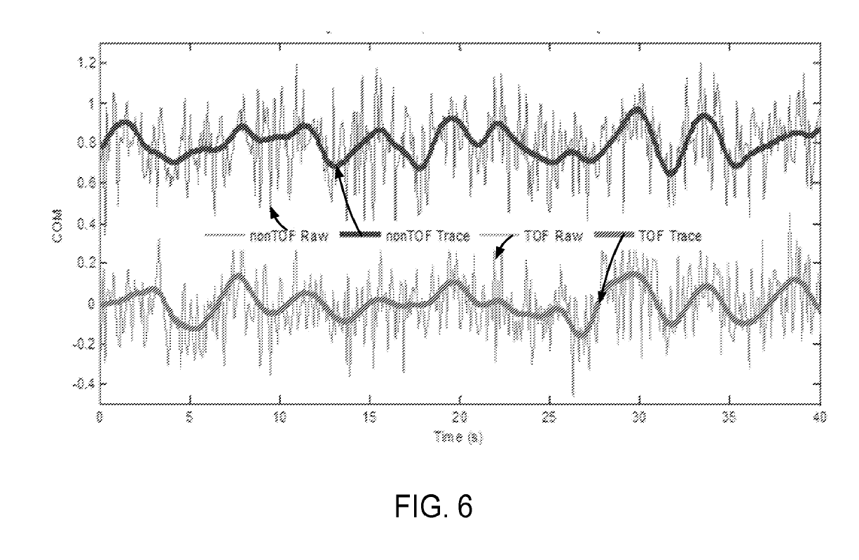

FIG. 6 shows raw TOF and non-TOF waveforms (mean corrected, patient 1) representing example respiratory signals computed using a COM algorithm in accordance with the present disclosure. Overlaid thereon are traces obtained by further processing the raw TOF and non-TOF waveforms. As shown in the figure, while the general breathing pattern is clearly observable in the raw waveforms, additional high- and low-frequency components are present. After analyzing the power spectral distribution, contributions caused by heart contractions were observed to center around frequencies between 0.75 and 1.16 Hz, while low-frequency contributions caused by respiratory motion were typically less than approximately 0.4 Hz. Appropriate filtering provided higher SNR.

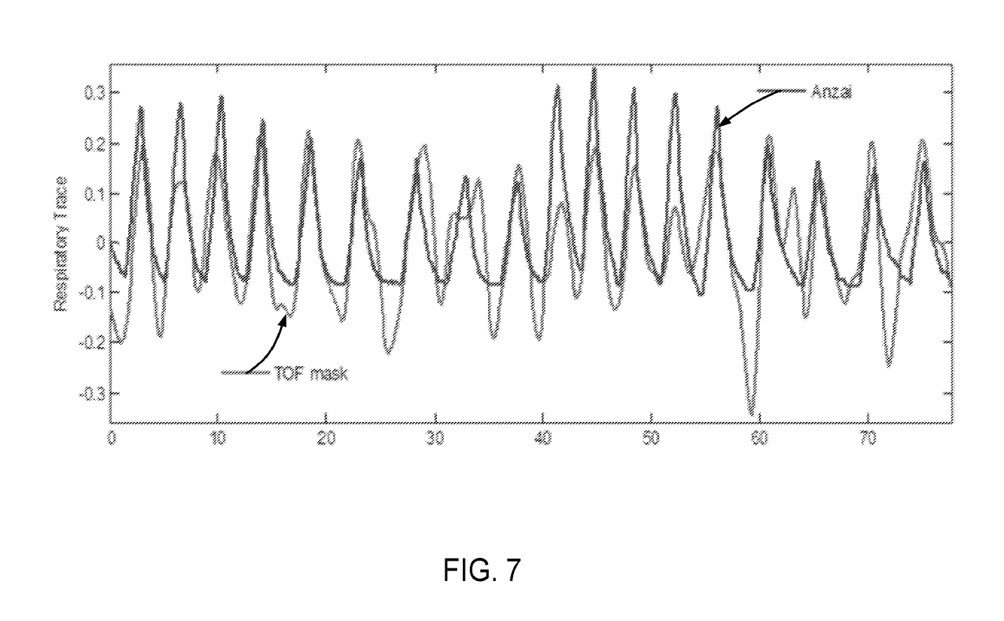

Respiratory traces derived from location-sensing sinogram of TOF PET were compared with Anzai traces. Generally, TOF traces showed a strong correlation with Anzai measurements. An example capturing one minute of data in the middle of a scan for patient 1 is shown in FIG. 7. A TOF trace, obtained in accordance with the present disclosure, was overlaid on an Anzai trace, wherein the mean was removed for the purpose of comparison. As is shown in the figure, the TOF trace showed great correlation with the Anzai trace, capturing variation in respiratory amplitude and frequency. In addition, even irregular respiration patterns were discernible. Also, a COD trace generated also showed good correlation with the Anzai trace.

In this study, respiratory traces were also computed from non-TOF-PET data with and without the masking technique, and compared with that from TOF-PET data with a location-sensing masking technique in order to demonstrate the improvement achieved by the present approach. Similarly, Anzai traces was used here as the ground truth. After comparison, both the TOF and nonTOF data-driven gating obtained good correlations with the Anzai gating.

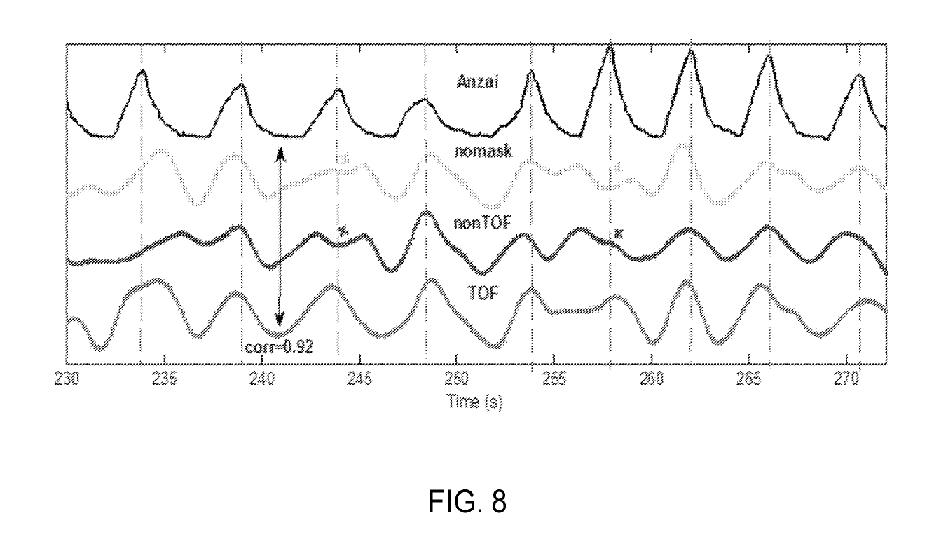

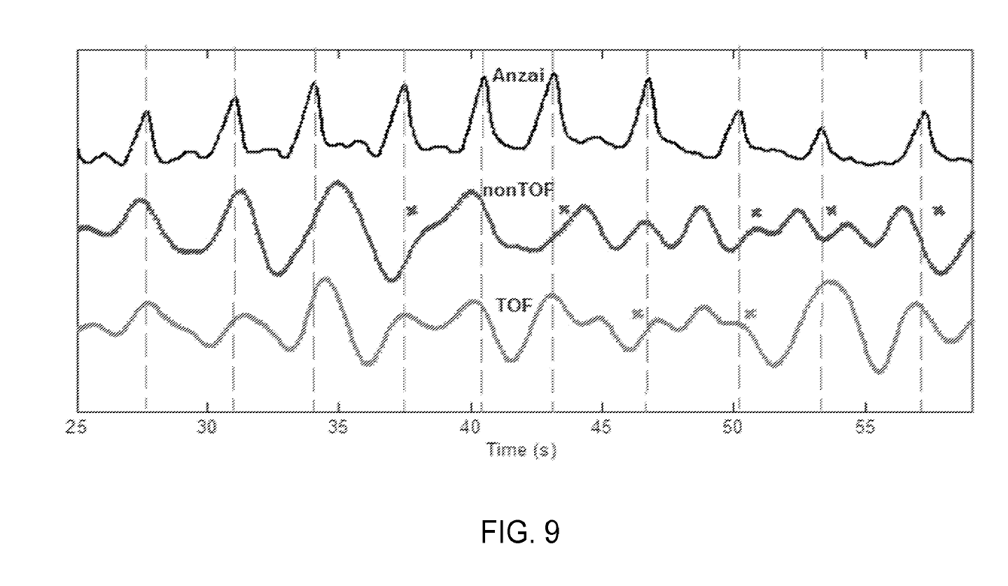

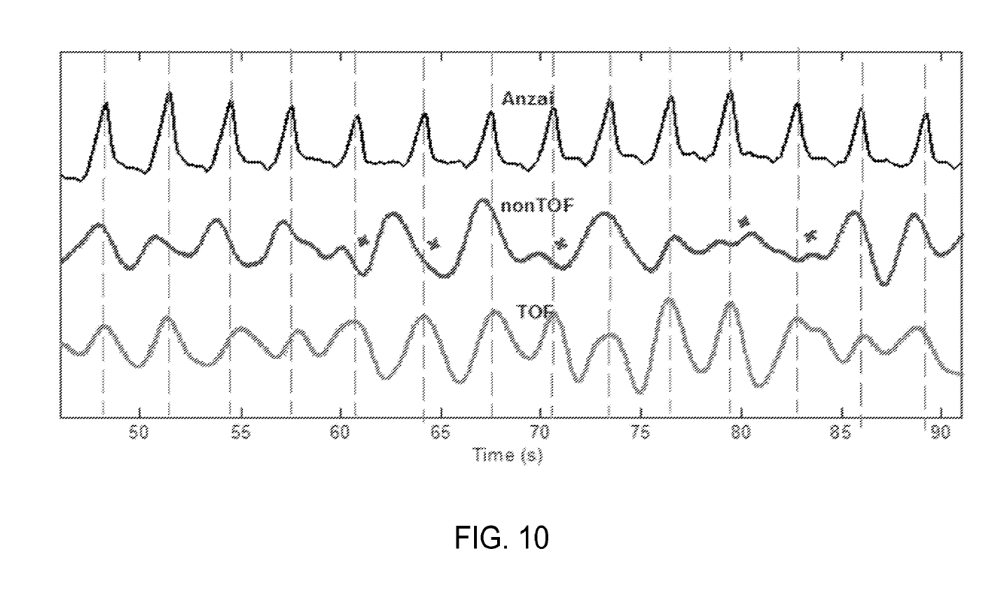

However, gating by nonTOF data, especially the gating without masking, was more likely to generate traces with discrepancies as compared to TOF-data based gating. An evident example is shown in FIG. 8, showing respiratory traces of patient 1 obtained with the four methods. Note that only a small segment of the data (42 sec out of 5 min) is plotted in FIG. 8, thus is only an illustrative display and not a complete result. By visual assessment, the TOF-based masking signal provided superior performance compared to non-TOF, while non-TOF gating without masking technique yielded a signal with the lowest accuracy. That is, gating with nonTOF data was more likely to generate a trigger with a large discrepancy compared to TOF gating, as shown in FIG. 8. As further examples, FIGS. 9 and 10 demonstrate the extracted respiratory traces of patients 2 and patient 3, respectively, obtained using the present approach and nonTOF PET with masking technique, in comparison with an Anzai waveform. Similarly to FIG. 8, FIGS. 9 and 10 demonstrate good performance of the present approach.

To quantitatively estimate the gating performance of the present approach and study the consistency of gating information, the error of trigger times and trace correlation with Anzai waveforms were computed for each patient from PET datasets. Individual triggers times were compared to determine the time difference Δt between Anzai and TOF (nonTOF) triggers. Triggers in various 50 s frames (patient 1 study) (except for invalid record) out of 300 seconds were calculated. The statistics of Δt are listed in Table 1.

TABLE 1
Accuracy of data-driven respiratory gating for patient 1

| | | Time | | | | | |
|---|---|---|---|---|---|---|---|
| Method | | 0-40 s | 60-139 s | 170-210 s | 230-272 s | Mean | Δt ≥400 ms % |
| nonTOF | TE^{a} | 0.26 ± 0.49 | 0.44 ± 0.64 | 0.32 ± 0.95 | 0.22 ± 0.84 | 0.31 ± 0.77 | 19.94 |
| no | TC^{b} | 0.65 | 0.63 | 0.55 | 0.59 | 0.61 | |
| mask | | | | | | | |
| nonTOf | TE | 0.05 ± 0.48 | 0.16 ± 0.57 | 0.20 ± 0.71 | 0.27 ± 0.61 | 0.17 ± 0.60 | 13.20 |
| mask | TC | 0.81 | 0.79 | 0.64 | 0.61 | 0.71 | |
| TOF | TE | 0.05 ± 0.41 | 0.01 ± 0.29 | 0.01 ± 0.45 | 0.06 ± 0.37 | 0.03 ± 0.38 | 3.14 |
| mask | TC | 0.83 | 0.91 | 0.89 | 0.81 | 0.86 | |

*^{a}TE: trigger error
^{b}TC: trace correlation

From the results listed in Table 1, both trigger errors and trace correlations were improved by using the present masking technique. For example, for frame 60-139 s, the trace correlation of Anzai signal and nonTOF data-driven signal without mask was 0.63, which is not a good estimation for respiratory motion. However, the use of masking technique helped to improve this figure of merit with the trace correlation of 0.79 and 0.91 for nonTOF mask and TOF mask respectively. Despite of the intra-patient variation in four respiratory durations, the TOF method yielded the best performance in terms of both correlation with Anzai waveform and detection accuracy of the respiratory triggers. The percentage of trigger with large (>400 ms) discrepancy was 13.20% when using masking techniques, but 3.14% for the present approach, illustrating a significant reduction by using TOF information.

Table 2 lists the statistic of trigger error and trace correlation with Anzai waveform for all 3 patients. Due to the inter-patient variation in breathing and biological features, the figures of merit are different for 3 patients. However, the advantage of TOF-based approach described herein over nonTOF-based method can be readily observed for each patient. Table 2 indicates that the incorporation of TOF information in sinogram space improved the performance of data-driven gating to varying degrees.

TABLE 2
Comparison of detection accuracy for all 3 patients

| Method | Pat | 1 | 2 | 3 |
|---|---|---|---|---|
| NonT | TE | 0.17 ± 0.60 | 0.32 ± 0.82 | 0.44 ± 0.71 |
| Of | TC | 0.71 | 0.60 | 0.67 |
| mask | Δt ≥ 400 ms % | 13.20 | 33.65 | 29.03 |
| TOF | TE | 0.03 ± 0.38 | 0.10 ± 0.35 | 0.06 ± 0.36 |
| mask | TC | 0.86 | 0.79 | 0.84 |
| | Δt ≥ 400 ms % | 3.14 | 7.14 | 4.17 |

*a: TE: trigger error
b: TC: trace correlation

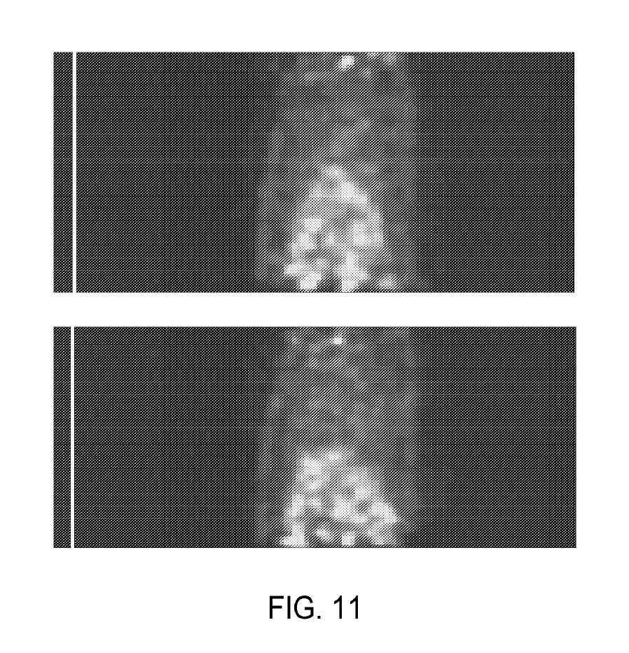

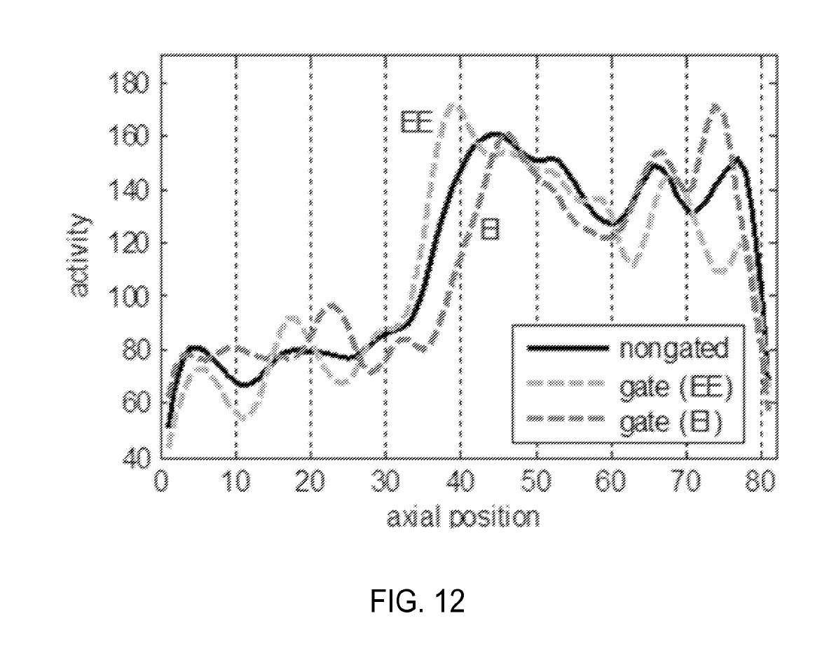

In addition, phase-gated reconstruction with 6 gates using gating information derived from PET data was performed. Non-gated and gated sagittal images obtained from patient 1 are shown in FIG. 11. In addition, FIG. 12 shows the 1D profile along the line FIG. 11 from the summed and gated reconstructed images in sagittal view. The gated images shown reveal respiratory motion of diaphragm and demonstrates an improved spatial contrast.

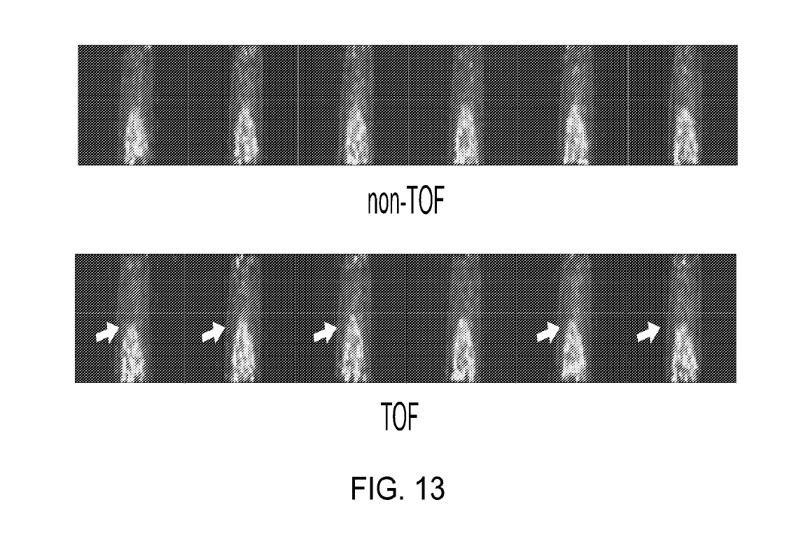

To demonstrate the improvement achieved by TOF information, TOF-gated PSF-OSEM images (gates 1-6) in coronal view are shown in FIG. 13, with nonTOF-gated images also being shown for comparison. Gate 1 is the phase of end-inspiration, corresponding to the peak of respiratory motion trace by either TOF or nonTOF-based method, and gate 4 is the phase of end-expiration, which corresponds to the valley. Visual comparison of the gated, non-attenuation-corrected PET images showed that the dome-of-liver boundary is more clearly defined for the gated. Moreover, motion of the diaphragm (top part of the liver) can be seen from these gated images.

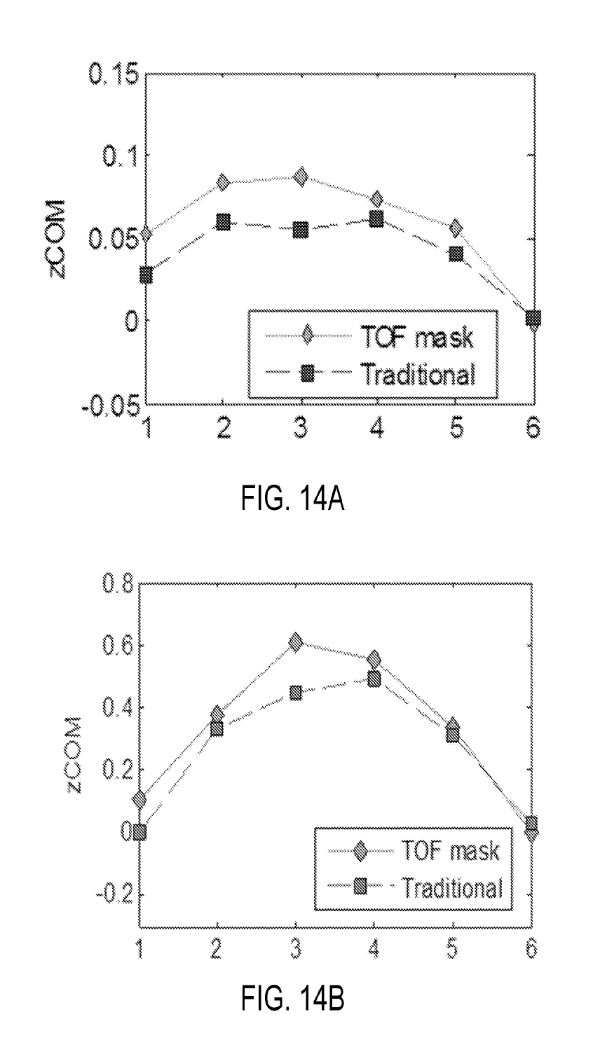

As may be appreciated from FIG. 13, the present TOF-gated approach outperforms the nonTOF-gated method in terms of visible motion shift. To determine the amount of displacement between each of the gated sinograms and images, the center of mass projected onto the z-axis of the scanner and image, respectively, was calculated for each gate. The range of displacement over the gates provided an indication of the amount of tumor motion in a lesion detection task using the present and other gating methods. The COM measurement calculated from PET sinograms and images are shown in FIGS. 14A and B. The range of motion measured on the gated sinogram was 0.8 mm and 0.6 mm and on the gated images was 6.1 mm and 4.5 mm for TOF and nonTOF based gating methods respectively.

In center-of-mass gating, the organs inside thorax may move along the axis during respiratory motion. Therefore by summing the sinograms events for each of the planes along the axial direction, the respiratory phase may be estimated without additional equipment. This approach may work under the assumption that all of the organs, such as heart, liver, lung and diaphragm may move with the same pattern during respiratory motion. Unfortunately, contrary to this ideal assumption, some features present in the field of view may not always contribute to the ‘center-of-mass’ of sinogram in the same way and hence adversely may affect the motion detection. Therefore, the present disclosure introduces a novel masking approach aimed at selectively including only those events that are likely to have been emitted from the specific organ, or part thereof, experiencing motion.

One masking technique involves excluding the planes where the specific organs may not be seen in the initial reconstruction. Another masking technique involves projecting a VOI into sinogram space to form a mask. Although more complicated, the latter approach may be able to exclude the event from features other than the defined VOI. However, in PET imaging, non-TOF-PET data cannot distinguish the exact location along an LOR of each event, and multiple events may contribute to the same element in the sinogram. Even though all the events from the specific organ may be kept, the events from unwanted tissues or organs, which may contribute to the same element of the sinogram, may also be kept. As described, the motion information from different features may contribute differently to the COM, and hence may reduce the signal to noise ratio of motion detection.

An advantage of the present approach for generating motion information is that, instead of applying mask in TOF sinogram space, the masking procedure described can be performed event by event using a list-mode data stream. Since sorting list-mode data into sinogram data may take both computation time and space, and the TOF-based masking allowed more ‘background’ data to be removed compared to non-TOF masking, the disclosed TOF-based method minimizes extra data processing as compared to other data-driven gating methods.

In addition, phase gating was demonstrated in the present disclosure, where each of the bins included an equal acquisition duration. However, the binning was based on the time passed since the beginning of the cycle, which may mean that maximum inspiration phases from two different cycles may be sorted together. This may result in inaccurate motion detection. As an alternative, it is envisioned that amplitude gating may be used to divide each breathing cycle into equal numbers of gates with respect to the amplitude of the respiratory signal. In this case, maximum inspiration phases from different breathing cycles may be sorted to the same gate of they reach the same depth of breathing. Therefore, amplitude binning may advantageously provide improved results.

Furthermore, tissue attenuation may degrade the accuracy of PET image, causing artifacts and non-uniform radioactive distribution. As such, attenuation correction may be of great significance to obtain the more accurate radioactive distribution information of subjects. Therefore, it is envisioned that various attenuation methods may be applied. An alternative to attenuation correction in 4D PET/CT may include dividing the CT acquisition into a series of motion-reduced frames and utilizing the gated CT images for the attenuation correction of the corresponding gated PET data obtained by present disclosed TOF-based method. The gated CT images may be obtained by both hardware-based gating techniques and CT data-driven respiratory gating method.

The present invention has been described in terms of one or more preferred embodiments, and it should be appreciated that many equivalents, alternatives, variations, and modifications, aside from those expressly stated, are possible and within the scope of the invention.

1. A method for generating motion information from positron emission tomography (PET) imaging, the method comprising:
receiving time-of-flight (TOF) data acquired using a PET system;
selecting, using at least one image reconstructed from the TOF data, a region of interest (ROI) having tissues subject to motion;
generating a TOF sinogram mask by projecting an image mask corresponding to the ROI into a sinogram space;
applying the TOF sinogram mask to a TOF sinogram, produced using the TOF data, to identify data in the TOF sinogram associated with motion; and
generating motion information using the data identified.

2. The method of claim 1, wherein the TOF data comprises list-mode data.

3. The method of claim 1, wherein the TOF sinogram is a three-dimensional (3D) TOF sinogram.

4. The method of claim 1, wherein the method further comprises selecting the ROI to be oriented in at least one of an axial direction and a transaxial direction.

5. The method of claim 1, wherein the method further comprises selecting the ROI to comprise at least one of a lung tissue, a heart tissue, a liver tissue, and a diaphragm tissue.

6. The method of claim 1, wherein the method further comprises performing a single slice rebinning (SSRB) algorithm using the data in the TOF sinogram associated with motion.

7. The method of claim 1, wherein the method further comprises applying a center of mass (COM) algorithm to generate the motion information.

8. The method of claim 1, wherein the method further comprises generating a respiratory waveform using the data in the TOF sinogram associated with motion.

9. The method of claim 8, wherein the method further comprises applying a band-pass filter to the respiratory waveform.

10. The method of claim 1, wherein the method further comprises using the motion information to perform a gated image reconstruction using the TOF data.

11. A system for generating motion information from positron emission tomography (PET) imaging, the system comprising:
a non-transitory computer readable medium having stored therein instructions for generating motion information;
a processor configured to execute the instructions to:
receive time-of-flight (TOF) data acquired from a subject using a PET system;
reconstruct at least one image using the TOF data;
generate an image mask based on a selection of a region of interest (ROI) having tissues subject to motion;
generate a TOF sinogram mask by projecting the image mask into a sinogram space;
apply the TOF sinogram mask to a TOF sinogram, produced using the TOF data, to identify data in the TOF sinogram associated with motion in the TOF sinogram; and
generate motion information using the identified data.

12. The system of claim 11, wherein the TOF data comprises list-mode data.

13. The system of claim 11, wherein the TOF sinogram is a three-dimensional (3D) TOF sinogram.

14. The system of claim 11, wherein the processor is further configured to analyze the at least one image reconstructed to select the ROI.

15. The system of claim 14, wherein the processor is further configured to select the ROI to be oriented in at least one of an axial direction and a transaxial direction.

16. The system of claim 11, wherein the processor is further configured to apply a center of mass (COM) algorithm to generate the motion information.

17. The system of claim 11, wherein the processor is further configured to generate a respiratory waveform using the data in the TOF sinogram associated with motion.

18. The system of claim 17, wherein the processor is further configured to apply a band-pass filter to the respiratory waveform.

19. The system of claim 11, wherein the processor is further configured to use the motion information to perform a gated image reconstruction using the TOF data.

20. The system of claim 11, wherein the processor is further configured to perform a single slice rebinning (SSRB) algorithm using the data in the TOF sinogram associated with motion.
